(19) United States
(12) Patent Application Publication
(10) Pub. No.: US 2018/0073088 A1
(43) Pub. Date: Mar. 15, 2018

(54) NOVEL HIGH-THROUGHPUT METHOD FOR QUANTIFICATION OF HBV CCCDNA FROM CELL LYSTATE BY REAL-TIME PCR

(71) Applicant: Hoffmann-La Roche Inc., Little Falls, NJ (US)

(72) Inventors: Waikwong Eng, Shanghai (CN); Zhaohu Lin, Shanghai (CN); Ning Qin, Shanghai (CN)

(73) Assignee: Hoffmann-La Roche Inc., Little Falls, NJ (US)

(21) Appl. No.: 15/638,128

(22) Filed: Jun. 29, 2017

Related U.S. Application Data
(63) Continuation of application No. PCT/EP2015/081259, filed on Dec. 28, 2015.

(30) Foreign Application Priority Data
Dec. 31, 2014 (CN) PCT/CN2014/095854
Dec. 28, 2015 (EP) 15823166.2

(51) Int. Cl. C12Q 1/70; C12Q 1/68

(52) U.S. Cl. CPC C12Q 1/706; C12Q 2600/16; C12Q 1/686; C12Q 1/6851

(57) ABSTRACT

The present invention provides (1) a set of novel DNA primers that can be used for high efficient real-time qPCR of Hepatitis B virus (HBV) circular DNA (cccDNA) and (2) a novel high throughput method for quantification of HBV cccDNA from cells using this set of novel DNA Primers by real-time qPCR. It also provides a use of this method in monitoring cccDNA level in experimentally infected liver cells and evaluation of therapeutic effect on HBV.

### FIELD OF THE INVENTION

The present invention relates to (1) a set of novel DNA primers that can be used for high efficient real-time qPCR of Hepatitis B virus (HBV) covalently closed circular DNA (cccDNA) and (2) a novel high throughput method for quantification of HBV cccDNA from cells using this set of novel DNA Primers by real-time qPCR. It also relates to the use of this method in monitoring cccDNA level in experimentally infected liver cells and evaluation of therapeutic effect on HBV.

### BACKGROUND OF THE INVENTION

Since cccDNA plays an important role in HBV biology, many experimental systems have been established for the study of potential drug candidates targeting cccDNA. In order to provide controllable cellular model of cccDNA, Ladner et al (Ladner, S. K., Otto, M. J., Barker, C. S., Zaifert, K., Wang, G. H., Guo, J. T., Seeger, C., King, R. W., Antimicrob Agents Chemother. 1997 August; 41(8):1715-20) have inserted HBV viral genome under the transcriptional control of tet promoter system. Furthermore, the length of insert is more than the length of the HBV whole genome, providing a sequence overlap over Nt (nucleotide) 1805-1900 (Cai et al, Antimicrob Agents Chemother. 2012 August; 56(8): 4277-88). This scheme allowed the use of eAg production as a surrogate marker for the cccDNA level and function. Finally, Guo et al (J. Virol. 2007, 81(22):12472) suggested that, upon further mutation of sAg gene in the above genetic background, the formation of viral DNA could not be used to form mature virus particle, thus following a pathway to be cycled into the nucleus to provide more precursors for cccDNA formation in a cell line named HepDES19. Because these modifications resulted in the controlled production of cccDNA, proxy measurement of cccDNA and the elimination of functional viral particles, the HepDES19 based cell model greatly facilitated the study of cccDNA and resulted in establishment of high throughput screening (HTS) assays using eAg as a surrogate marker for cccDNA (Antimicrob Agents Chemother. 2012 August; 56(8): 4277-88).

However, cccDNA is a direct target of interest for HBV disease treatment. Direct measurement of cccDNA in cell lines, specifically in HepDES19 cells, is thus of significance in the activity evaluation on cccDNA production and stability.

Measurement of cccDNA by conventional techniques such as Southern hybridization cannot be used as a high throughput method due to its low sensitivity or throughput. He (BBRC 295 (2002) 1102-1107, US patent application: 20040058314), Shao et al (CN 101503740 B) and Lu et al (CN 101285105B) have previously reported qPCR based cccDNA quantification assay methods. While the detailed qPCR methods may be different, all of the methods require extraction of HBV DNA before analysis. DNA extraction is complex and removes a critical component of the system important for qPCR. Thus, when such DNA extraction is involved, it is commonly controlled by using equal amount of starting materials, such as cell number. Although such control provides indications of how to attempt to equalize results, they do not comprise a real experimental control strictly speaking.

Because of the inclusion of DNA purification steps, cccDNA direct measurement by qPCR cannot be scaled up into a high throughput screen format (Hirt, B, J. Mol. Biol. 1967; 26, 365-9). In order to provide a better method that can enable high throughput measurement of cccDNA in all sample sources, particularly those from HepDES19 cellular model system, a new robust screening scheme is needed.

Many people have described methods of directly measure specific forms of certain nucleic acid in cell lysate samples without pre-purification of DNA (Yang K. et al, Gene. 2013 Dec. 1, 531(2):199-204; Kumar J S et al, Mol Cell Probes. 2014 Apr. 13; Pang Z et al, PLoS One. 2014 Apr. 21, 9(4): e95635.). These approaches significantly increased assay efficiency. However, these approaches cannot be applied to cccDNA qPCR methods described by He, Shao and Lu. Based on the primer sequence information from the referenced methods of theirs, all of their reverse primers reside within the sequence range of upstream of Nt-1990 of HBV as they can be easily used in the q-PCR assay. Because of this, all of those qPCR based methods will amplify DNA signal from the chromosome insert of 1.1×HBV genome (Guo et al, J Virol. 2007 November, 81(22):12472-84), thus failing to selectively amplify cccDNA in HepDES19 cells. Furthermore, direct lysis followed by qPCR requires higher specificity of the primers involved to distinguish cccDNA from other forms of HBV DNA, such as relaxed circular DNA (rcDNA) and HBV genome inserted in cells.

In summary, there is no sensitive and specific HTS direct assay available to quantitate cccDNA level in HBV infected cells, in particular, HepDES19, without pre-extracting HBV DNA. Thus, there is a need for a method to quantitate cccDNA with high sensitivity, high throughput and high specificity. The object of the present invention, as set forth herein, is to meet these needs.

### BRIEF SUMMARY OF THE INVENTION

This invention is to develop a one-step cccDNA quantitative HTS (High Throughput Screening) method by direct real-time PCR from HBV infected cells, in particular, HepDES19 cell without prior DNA extraction and purification.

We have designed a set of cccDNA specific primers. This novel set of primers has been proven to be cccDNA specific in amplifying DNA fragments from HBV cccDNA but not from the genomic DNA or chromosome inserted HBV DNA. The specificity for cccDNA of these primers eliminates the need for DNA extraction and cccDNA purification steps and allows us to quantitate cccDNA in microtiter plate assay format at HTS mode.

One embodiment of present invention is a reverse primer comprising at its extreme 3′ end a sequence recognizing at least 16 consecutive within the nucleotide sequence of the Hepatitis B Virus (HBV) genome (SEQ ID NO: 1).

Another embodiment of present invention is a primer pair consisting of a reverse primer having the significances as described above and a universal forward primer, wherein the length of the universal forward primer is 16 to 200 nucleotides, 17 to 35 nucleotides, 18 to 30 nucleotides, 21 to 30 nucleotides, 24 to 30 nucleotides or 21 to 24 nucleotides, and recognizes upstream of DR2 of the HBV genome.

Another embodiment of present invention is a probe for detecting the extracted DNA or the lysate of one-step qPCR assay, wherein the length of the probe is 16 to 200 nucleotides, 17 to 35 nucleotides, 18 to 30 nucleotides, 21 to 30 nucleotides, 24 to 30 nucleotides or 21 to 24 nucleotides.

The present invention relates to a combination comprising of a reverse primer and a probe, wherein the reverse primer and the probe have the significances as described above.

The present invention relates to a method for detecting HBV cccDNA by PCR, comprising of using the HBV genome (SEQ ID NO: 1) as the template, using a primer pair of a reverse primer and a universal forward primer or using a combination comprising of a reverse primer and a probe, wherein the reverse primer, the universal forward primer and the probe have the significances as described above.

The present invention relates to a method used in evaluation of therapeutic effect on HBV, wherein the method has the significances as described above.

The present invention relates to the use of a reverse primer, a primer pair of a reverse primer and a universal forward primer or a combination comprising of a reverse primer and a probe for evaluation of therapeutic effect on HBV, wherein the reverse primer, the universal forward primer and the probe have the significances as described above.

The present invention relates to the use of a reverse primer, a primer pair of a reverse primer and a universal forward primer or a combination comprising of a reverse primer and a probe in preparation of a detecting agent for evaluation of therapeutic effect on HBV, wherein the reverse primer, the universal forward primer and the probe have the significances as described above.

The present invention relates to the use of an internal reference gene as a normalization control. It comprises the use of primers for gene(s) on genomic DNA as qPCR quantification controls.

The preservation of proportion of all DNA components in the invention allows for such quantification control.

The present invention relates to a kit for evaluation of therapeutic effect on HBV, comprising of a reverse primer, a primer pair, or a combination, wherein the reverse primer, the primer pair, and the combination have the significances as described above.

The present invention also relates to a multiplexing kit for evaluation of therapeutic effect on HBV, comprising of a reverse primer, a primer pair, in combination with a reference control gene primers and a pair of probes for HBV DNA and for reference gene, wherein the reverse primer and the primer pair have the significances as described above.

### Definitions

Unless defined otherwise, all technical and scientific terms used herein have the same meaning as commonly understood by one of ordinary skill in the art to which this invention pertains. Although essentially any methods and materials similar to those described herein can be used in the practice or testing of the present invention, only exemplary methods and materials are described. For purposes of the present invention, the following terms are defined below.

The term “nucleotide” in addition to referring to the naturally occurring ribonucleotide or deoxyribonucleotide monomers, shall herein be understood to refer to related structural variants thereof, including derivatives and analogs, that are functionally equivalent with respect to the particular context in which the nucleotide is being used (e.g., hybridization to a complementary base), unless the context clearly indicates otherwise.

The term “identity” in the context of two or more nucleic acids or polypeptide sequences, refers to two or more sequences or subsequences that are the same. Sequences are “substantially identical” to each other if they have a specified percentage of nucleotides or amino acid residues that are the same (e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% identity over a specified region), when compared and aligned for maximum correspondence over a comparison window, or designated region as measured using one of the following sequence comparison algorithms or by manual alignment and visual inspection. These definitions also refer to the complement of a test sequence. Optionally, the identity exists over a region that is at least about 50 nucleotides in length, or more typically over a region that is 100 to 500 or 1000 or more nucleotides in length.

The term “PCR” as used herein refers to polymerase chain reaction.

The term “qPCR” generally refers to the PCR technique known as real-time quantitative polymerase chain reaction, quantitative polymerase chain reaction or kinetic polymerase chain reaction. This technique simultaneously amplifies and quantifies target nucleic acids using PCR wherein the quantification is by virtue of an intercalating fluorescent dye or sequence-specific probes which contain fluorescent reporter molecules that are only detectable once hybridized to a target nucleic acid.

The term “primer” as used herein refers to an oligo nucleotide DNA capable of serving as DNA replication reaction initiator in a qPCR reaction.

The term “probe” as used herein refers to an oligo nucleotide with fluorescence label capable of changing fluorescence intensity changes correlating to the increase of level of complementary DNA present in the reaction.

The term “Cp” refers to a value that allows quantification of input target nucleic acids. The Cp value can be determined according to the second-derivative maximum method (Van Luu-The, et al., “Improved real-time RT-PCR method for high-throughput measurements using second derivative calculation and double correction,” BioTechniques, Vol. 38, No. 2, February 2005, pp. 287-293). In the second derivative method, a Cp corresponds to the first peak of a second derivative curve. This peak corresponds to the beginning of a log-linear phase. The second derivative method calculates a second derivative value of the real-time fluorescence intensity curve, and only one value is obtained. The original Cp method is based on a locally defined, differentiable approximation of the intensity values, e.g., by a polynomial function. Then the third derivative is computed. The Cp value is the smallest root of the third derivative. The Cp can also be determined using the fit point method, in which the Cp is determined by the intersection of a parallel to the threshold line in the log-linear region (Van Luu-The, et al., BioTechniques, Vol. 38, No. 2, February 2005, pp. 287-293). These computations are easily carried out by any person skilled in the art.

The term “extraction” as used herein refers to the purification of extra chromosomal DNA according to the method of modified Hirt Procedure (Arad U, Biotechniques. 1998 May; 24(5):760-2; Hirt, B, J. Mol. Biol. 1967; 26, 365-9).

The term “conventional” refers to experimental methods and materials based on well accepted published references in the field. To avoid ambiguity, “Conventional Group” and “Conventional” in the Figures have different meanings, which is illustrated hereinbelow in the abbreviation part.

The term “one-step qPCR” refers to experimental procedure where cell materials were lysed and directly subjected to qPCR analysis.

The term “Z factor” is a measure of assay statistical reproducibility. It has been proposed for use in high-throughput screening (where it is also known as Z-prime, and commonly written as Z′) to judge whether the response in a particular assay is large enough to warrant further attention. The Z-factor is defined in terms of four parameters: the means and standard deviations of both the positive (p) and negative (n) controls (μₚ, σₚ, μₙ and σₙ). Given these values, the Z-factor is defined as:

$$Z \text{-} factor = 1 - \frac{3 ( {σ}_{p} + {σ}_{n} )}{\vert {μ}_{p} - {μ}_{n} \vert} .$$

The term “TAMRA” refers to 5-carboxy-tetramethylrhodamine N-succinimidyl ester as a dye. Dye Substitutes: When seeking dye alternatives, the following criteria are important: 1) The excitation and detection wavelength are compatible with the instrument light source and detection system. 2) For probes, the quencher effectively absorbs light at the emission wavelength of the reporter. 3) The higher the extinction coefficient the brighter the dye, which contributes to sensitive detection.

The term “BHQ2” refers to a dye quencher Blackhole Quencher-2 as a quencher. Quenchers: Quenching molecules are typically placed at the 3′ end of single molecule probes. Quenchers may be fluorescent (TAMRA™) or nonfluorescent molecules (DABCYL, Black Hole Quencher® (BHQ®). For optimal performance, the quencher's absorbance spectrum should match the reporter's emission spectrum as closely as possible. Commonly used quenchers are DABCYL and TAMRA. Commonly used dark quenchers include BlackHole Quenchers™ (BHQ), Iowa Black™ and BlackBerry™ Quencher 650 (BBQ-650). Commonly used pairs are dye 6-FAM/JOE/TET with quencher BHQ-1/TAMRA, dye TAMRA with quencher BHQ-2.

### BRIEF DESCRIPTION OF THE FIGURE(S)

FIG. 1: FIG. 1 depicts the integrated HBV DNA in HepDES19 stable cells and the scheme of the design principle. The integrated HBV DNA in HepDES19 cells is composed of 1.1 fold overlength HBV genome, beginning from nucleotide 1805, placed downstream of a tet CMV promoter. The HBV Nt positions are according to the Galibert nomenclature (Galibert, F, et al, Nature 281:646-650). As shown in the figure, the position of the forward primer (F) sequence is within the single stranded portion of the double-stranded relaxed circular DNA (rcDNA). Because of this, reverse reaction product in rcDNA cannot be used as template by the forward primer in the next cycle of PCR. Furthermore, the probe in the qPCR design is downstream of the nick in rcDNA. The presence of the physical break prevents the forward reaction from moving into the probe positions in rcDNA. In cccDNA, there is no nick structure, allowing the probes to be cleaved by polymerase, causing the fluorescence to increase. These two factors explain the qPCR scheme in terms of selectivity for cccDNA over rcDNA. However, in HepDES19 cells, there is also the chromosomal 1.1 fold HBV genomic insert present. The 3′ terminal redundancy sequence for this 1.1 fold over-length HBV genome is from Nt 1805 to 1990.

We firstly found if reverse primers were designed from upstream of 1990, the chromosomal HBV DNA insert would be amplified in the qPCR assay, making the extraction of viral DNA necessary for cccDNA quantification. This explains why it is necessary to extract viral DNA when using conventional reverse primers (Takkenberg et al, Methods in Molecular Biology, vol. 903, DOI 10.1007/978-1-61779-937-2_7). We surprisingly found that by designing reverse primers downstream of 1990, the new reverse primers were situated on the left side of the forward primer in the figure (pointing away from each other), the genomic HBV DNA insert was not amplified in the qPCR assay, and the new reverse primers was still pointing at the forward primer on cccDNA, resulting in productive qPCR reaction due to the circular nature of cccDNA.

FIG. 2: FIG. 2 depicts qPCR assay using conventional cccDNA primer pairs for extracted viral genome DNA and using one-step qPCR assay.

FIGS. 3a and 3b: FIG. 3a and FIG. 3b depict the design and test of new cccDNA primers for detecting the extracted DNA or the direct lysate without extraction (one-step qPCR assay) from HepDES19.

FIGS. 4a and 4b: FIGS. 4a and 4b depict the design and test of new cccDNA probes for detecting the extracted DNA or the direct lysate of one-step assay without extraction (one-step qPCR assay) from HepDES19.

FIG. 5: FIG. 5 provides size comparison of PCR products from pHBV1.3 positive control plasmid (for pHBV1.3 positive control plasmid, please refer to J. Virol. 2007 Sep.; 81(18): 10072-80. Epub 2007 Jul. 3) and one-step qPCR assay by DNA agarose gel.

FIG. 6: FIG. 6 provides the confirmation of the specificity of cccDNA primers using pHBV1.3 positive plasmid and HBV DNA controls by qPCR assay.

FIG. 7: FIG. 7 provides the confirmation of the specificity of cccDNA primer pairs either with or without Plasmid Safe ATP-dependent DNase (PSAD, which can cleave non-circle DNA, purchased from Epicentre, Madison, Wis., USA, Catalogue number: E3101K) pre-treatment followed by qPCR assay.

FIGS. 8a and 8b: FIGS. 8a and 8b provides the confirmation of the specificity of cccDNA primers using the supernatant of HepG2.2.15, HepDE19 and HepDES19 cells by qPCR assay.

FIG. 9: FIG. 9 depicts the test of the level of cccDNA in 96 well microtiter plate (Corning 3599).

FIG. 10: FIG. 10 depicts the design of new reverse primers and comparison of their ability for detection of HBV cccDNA.

### DETAILED DESCRIPTION OF THE INVENTION

The present invention provides a novel high-throughput method for quantification of HBV cccDNA from cell lysate by real-time PCR and its use in monitoring cccDNA level in infected liver cells and evaluation of therapeutic effect on HBV.

One embodiment of present invention is (i) a reverse primer comprising at its extreme 3′ end a sequence recognizing at least 16 consecutive sequence within the nucleotide sequence of the Hepatitis B Virus (HBV) genome (SEQ ID NO: 1).

A further embodiment of present invention is (ii) a reverse primer comprising at its extreme 3′ end a sequence recognizing at least 16 consecutive sequence within nucleotide sequence of HBV genome from 1996 to nucleotide 2278 of SEQ ID NO: 1.

Another embodiment of present invention is (iii) a reverse primer having the significances given in embodiment (i) or (ii), wherein the length of the reverse primer is 16 to 200 nucleotides, 17 to 35 nucleotides, 18 to 30 nucleotides, 21 to 30 nucleotides, 24 to 30 nucleotides or 21 to 24 nucleotides.

Another embodiment of present invention is (iv) a reverse primer, preferably at its extreme 3′ end nucleotides, in the 5′ to 3′ direction, comprising the sequence selected from:

SEQ ID NO: 10: R0 2005-2025 (reverse complement)
5′-AAGGCTTCCCGATACAGAGCT-3′;
SEQ ID NO: 11: R1 1996-2016 (reverse complement)
5′-CGATACAGAGCTGAGGCGGTA-3′;
SEQ ID NO: 12: R2 2001-2021 (reverse complement)
5′-CTTCCCGATACAGAGCTGAGG-3′;
SEQ ID NO: 13: R3 20452046-2067 (reverse
complement)
5′-CTGAGTGCAGTATGGTGAGGTG-3′;
SEQ ID NO: 14: R4 2097-2118 (reverse complement)
5′-CCCACCCAGGTAGCTAGAGTCA-3′;
SEQ ID NO: 15: R5 2129-2152 (reverse complement)
5′-TAGGTCTCTAGACGCTGGATCTTC-3′;
SEQ ID NO: 16: R6 2183-2206 (reverse complement)
5′-CCACAAGAGTTGCCTGAACTTTAG-3′;
SEQ ID NO: 17: R7 2188-2210 (reverse complement)
5′-GAAACCACAAGAGTTGCCTGAAC-3′;
SEQ ID NO: 18: R8 2210-2233 (reverse complement)
5′-TCCAAAAGTGAGACAAGAAATGTG-3′;
SEQ ID NO: 19: R9 2249-2272 (reverse complement)
5′-CACTCCGAAAGACACCAAATACTC-3′;
SEQ ID NO: 20: R10 2251-2274 (reverse complement)
5′-CACACTCCGAAAGACACCAAATAC-3′;
SEQ ID NO: 21: R11 2257-2278 (reverse complement)
5′-AATCCACACTCCGAAAGACACC-3′;
and
SEQ ID NO: 24: R4 (30 bp) 2097-2126 (reverse
complement)
5′-AATTAACACCCACCCAGGTAGCTAGAGTCA-3′.

Another embodiment of present invention is (v) a primer pair, consisting of a reverse primer and a universal forward primer, wherein the reverse primer has the significances as defined herein before, the length of the universal forward primer is 16 to 200 nucleotides, 17 to 35 nucleotides, 18 to 30 nucleotides, 21 to 30 nucleotides, 24 to 30 nucleotides or 21 to 24 nucleotides, and the universal forward primer recognizes upstream of DR2 of the HBV genome.

A further embodiment of present invention is (vi) a primer pair consisting of a reverse primer and a universal forward primer, wherein the reverse primer has the significances as defined herein before; the length of the universal forward primer is 16 to 200 nucleotides, 17 to 35 nucleotides, 18 to 30 nucleotides, 21 to 30 nucleotides, 24 to 30 nucleotides or 21 to 24 nucleotides, and the universal forward primer has at least 80% sequence identity with the nucleotide sequence of SEQ ID NO: 5 from nucleotide 1528 to nucleotide 1548.

A further embodiment of present invention is (vii) a primer pair consisting of a reverse primer and a universal forward primer, wherein the reverse primer has the significances as defined herein before; the length of the universal forward primer is 16 to 200 nucleotides, 17 to 35 nucleotides, 18 to 30 nucleotides, 21 to 30 nucleotides, 24 to 30 nucleotides or 21 to 24 nucleotides, and the universal forward primer recognizes upstream of DR2 of the HBV genome and has at least 80% sequence identity with the nucleotide sequence of SEQ ID NO: 5 from nucleotide 1528 to nucleotide 1548.

A further embodiment of present invention is (viii) a primer pair consisting of a reverse primer and a universal forward primer, wherein the reverse primer has the significances as defined above; the length of the universal forward primer is 16 to 200 nucleotides, 17 to 35 nucleotides, 18 to 30 nucleotides, 21 to 30 nucleotides, 24 to 30 nucleotides or 21 to 24 nucleotides, and the universal forward primer in the 5′ to 3′ direction comprises SEQ ID NO: 5.

Another embodiment of present invention is (ix) a probe for detecting the extracted DNA or the lysate of one-step qPCR assay, wherein the length of the probe is 16 to 200 nucleotides, 17 to 35 nucleotides, 18 to 30 nucleotides, 21 to 30 nucleotides, 24 to 30 nucleotides or 21 to 24 nucleotides. In particular, the present invention also relates to a probe for detecting the extracted DNA or the lysate of one-step qPCR assay from HepDES19, wherein the length of the probe is 16 to 200 nucleotides, 17 to 35 nucleotides, 18 to 30 nucleotides, 21 to 30 nucleotides, 24 to 30 nucleotides or 21 to 24 nucleotides.

A further embodiment of present invention is (x) a probe for detecting the extracted DNA or the lysate of one-step qPCR assay, wherein the probe has at least 80% sequence identity with a sequence comprising a part of a sequence selected from SEQ ID NO: 6, SEQ ID NO: 7, SEQ ID NO: 8 and SEQ ID NO: 9.

Further embodiment of present invention is (xi) a probe has at least 80% sequence identity with SEQ ID NO: 6, SEQ ID NO: 7, SEQ ID NO: 8 and SEQ ID NO: 9, and other technical features have the significances as defined above.

Further embodiment of present invention is (xii) a probe, in the 5′ to 3′ direction, has the sequence selected from SEQ ID NO: 6, SEQ ID NO: 7, SEQ ID NO: 8 and SEQ ID NO: 9, and other technical features have the significances as defined above.

A further embodiment of present invention is (xiii) a probe of P0, P1, P2 or P3, wherein

P0 is 5′-dye+SEQ ID NO: 6+quencher-3′;

P1 is 5′-dye+SEQ ID NO: 7+quencher-3′;

P2 is 5′-dye+SEQ ID NO: 8+quencher-3′;

P3 is 5′-dye+SEQ ID NO: 9+quencher-3′.

A further embodiment of present invention is (xiv) a probe having at least 80% sequence identity with a sequence comprising a part of a sequence selected from SEQ ID NO: 6, SEQ ID NO: 7, SEQ ID NO: 8 and SEQ ID NO: 9, wherein the dye is 6-FAM, JOE or TET while the quencher is BHQ-1 or TAMRA, or the dye is TAMRA while the quencher is BHQ-2.

Another embodiment of present invention is (xv) a combination comprising of a reverse primer and a probe, wherein the reverse primer is R0, R1, R2, R3, R4, R5, R6, R7, R8, R9, R10, R11 or R4 (30 bp); and the probe is P0, P1, P2 or P3.

A further embodiment of present invention is (xvi) a combination comprising of a reverse primer and a probe, wherein the reverse primer is R3, R4, R5 or R4 (30 bp); and the probe is TAMRA+SEQ ID NO: 6+BHQ2 or TAMRA+SEQ ID NO: 8+BHQ2.

Another embodiment of present invention is (xvii) a method for detecting HBV cccDNA by PCR, comprising of using the HBV genome (SEQ ID NO: 1) as the template, and using a primer pair of a reverse primer and a universal forward primer or a combination comprising of a reverse primer and a probe; the reverse primer, the universal forward primer and the probe have the significances as defined above.

Another embodiment of present invention is (xviii) a method for detecting HBV cccDNA by PCR, comprising using the HBV genome (SEQ ID NO: 1) as the template, and using a primer pair of a reverse primer and a universal forward primer or a combination comprising of a reverse primer and a probe; wherein the PCR is real-time PCR; the reverse primer, the universal forward primer and the probe have the significances as defined above.

Another embodiment of present invention is (xix) a method for detecting HBV cccDNA by PCR, comprising of using the HBV genome (SEQ ID NO: 1) as the template, and using a primer pair of a reverse primer and a universal forward primer or a combination comprising of a reverse primer and a probe; wherein the method is a high-throughput method; the reverse primer, the universal forward primer and the probe have the significances as defined above.

Another embodiment of present invention is (xx) a one-step cccDNA detection method by real-time PCR without pre DNA extraction and purification, wherein the method comprises the following procedure:

(1) treating cells with compounds to be tested;

(2) harvesting cells after treatment;

(3) adding the lysis buffer to the harvested cells; and

(4) carrying out the real time PCR having the significances as defined above.

A further embodiment of present invention is (xxi) a one-step cccDNA detection method by real-time PCR without pre DNA extraction and purification according to embodiment (xx), wherein the lysis buffer is selected from commercially available lysis buffers such as RIPA buffer: 50 mM Tris-HCl (pH 7.5), 150 mM NaCl, 1% Triton X-100, 0.5% sodium deoxycholate, 0.1% SDS and 5 mM EDTA; NP-40 buffer: 50 mM Tris-HCl (pH 8.0), 150 mM NaCl, 0.5% NP-40 substitute and 5 mM EDTA; and DSP buffer: 40 mM HEPES (pH 7.5), 120 mM NaCl, 1% Triton X-100, 1 mM EDTA, 10 mM β-glycerophosphate and 50 mM NaF; and other technical features have the significances as defined above.

A further embodiment of present invention is (xxii) one-step cccDNA detection method by real-time PCR without pre DNA extraction and purification according to embodiment (xx), wherein the lysis buffer is from Invitrogen and the catalogue number is AM8723; and other technical features have the significances as defined above.

A further embodiment of present invention is (xxiii) one-step cccDNA detection method by real-time PCR without pre DNA extraction and purification according to embodiment (xx), wherein the cells are treated for 30 minutes to 2 hours at a temperature between 50° C. and 75° C. in an oven, 20-100 μL of the lysis buffer is added per well in 96 well plate and then real time PCR is conducted at a temperature between 0° C. and 25° C., and other technical features have the significances as defined above.

Another embodiment of present invention is (xxiv) a method used in evaluation of therapeutic effect on HBV comprising using the method having the significances as defined above.

Another embodiment of present invention is (xxv) the use of a reverse primer, a primer pair of a reverse primer and a universal forward primer or a combination comprising of a reverse primer and a probe for evaluation of therapeutic effect on HBV, wherein the reverse primer, the universal forward primer and the probe have the significances as defined above.

Another embodiment of present invention is (xxvi) the use of a reverse primer, a primer pair of a reverse primer and a universal forward primer or a combination comprising of a reverse primer and a probe in preparation of a detecting agent for evaluation of therapeutic effect on HBV, wherein the reverse primer, the universal forward primer and the probe have the significances as defined above.

Another embodiment of present invention is (xxvii) is a kit for evaluation of therapeutic effect on HBV, comprising of a reverse primer, a primer pair of a reverse primer and a universal forward primer or a combination comprising of a reverse primer and a probe, wherein the reverse primer, the universal forward primer and the probe have the significances as defined above.

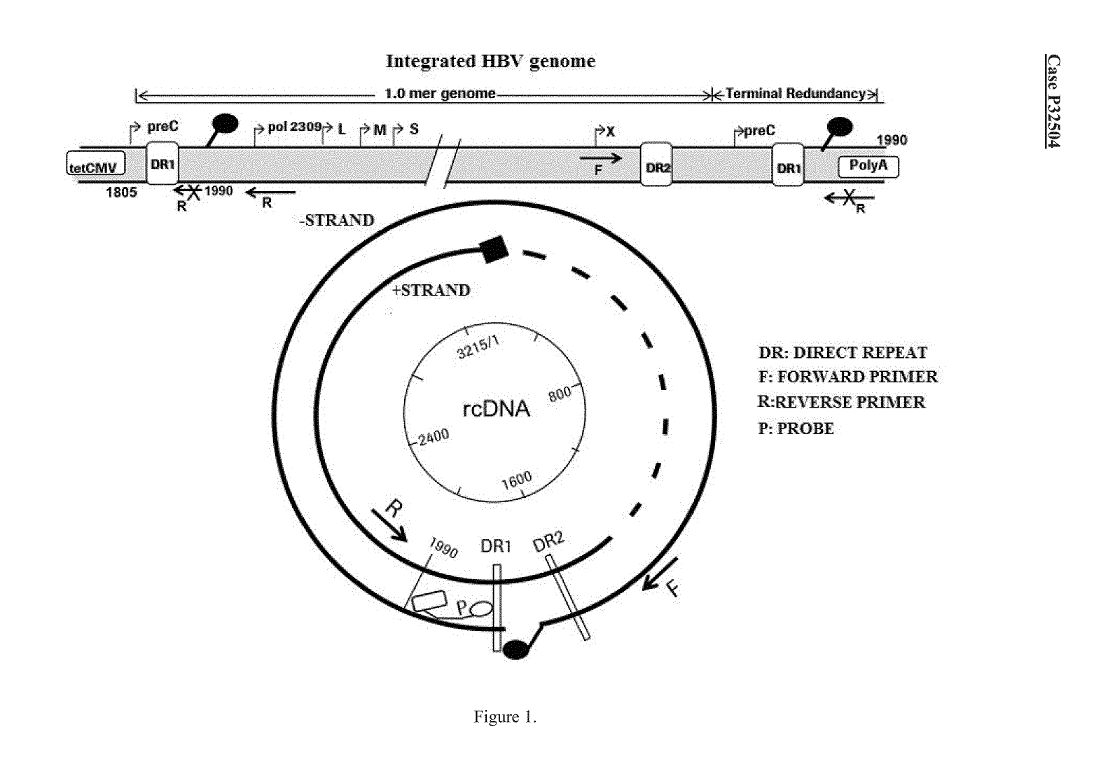

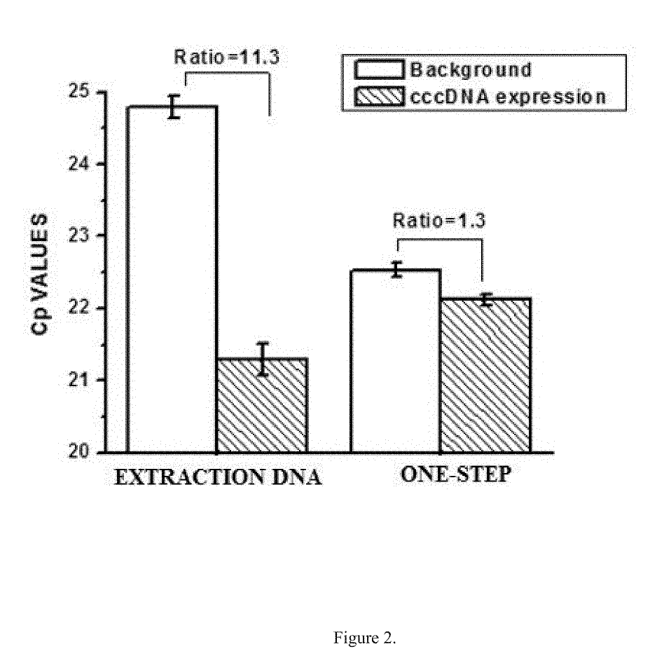

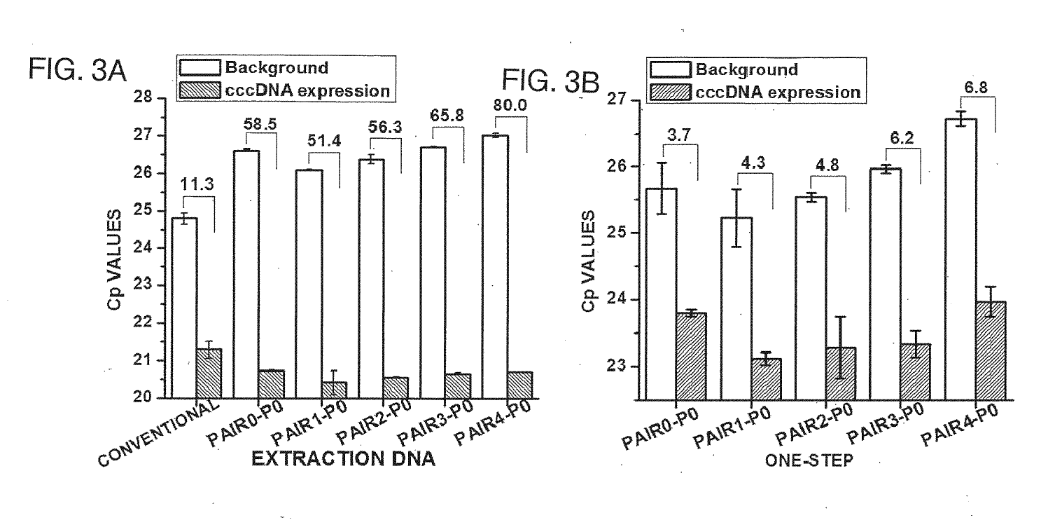

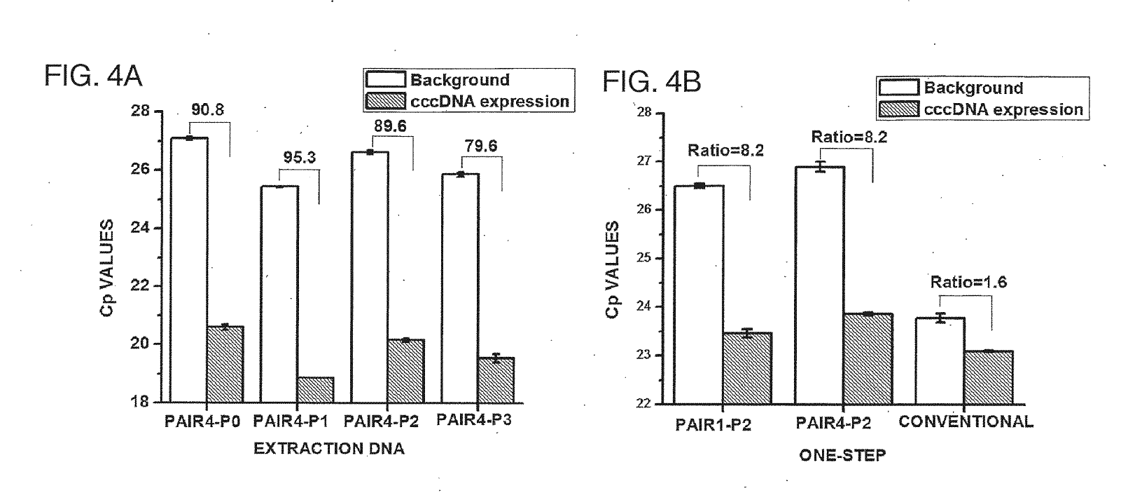

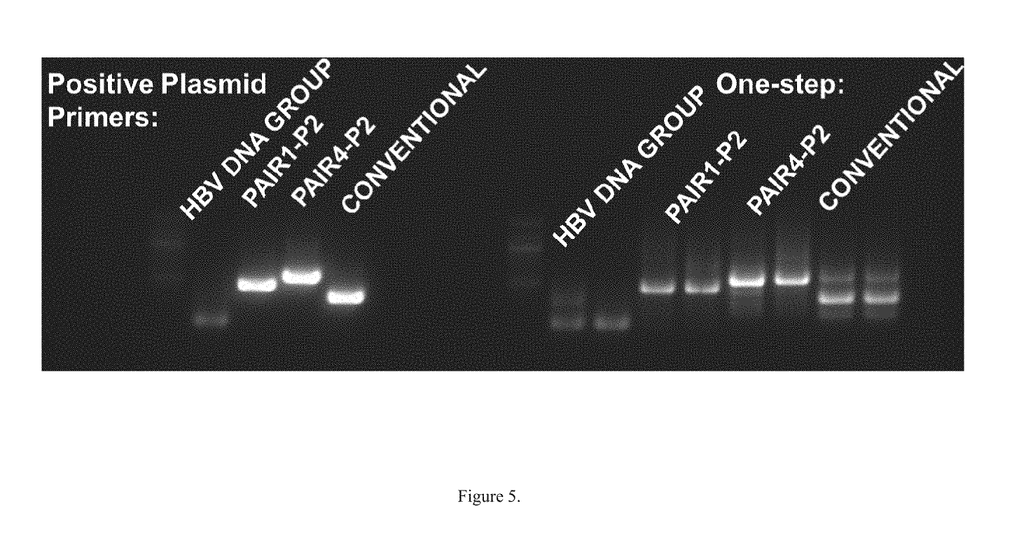

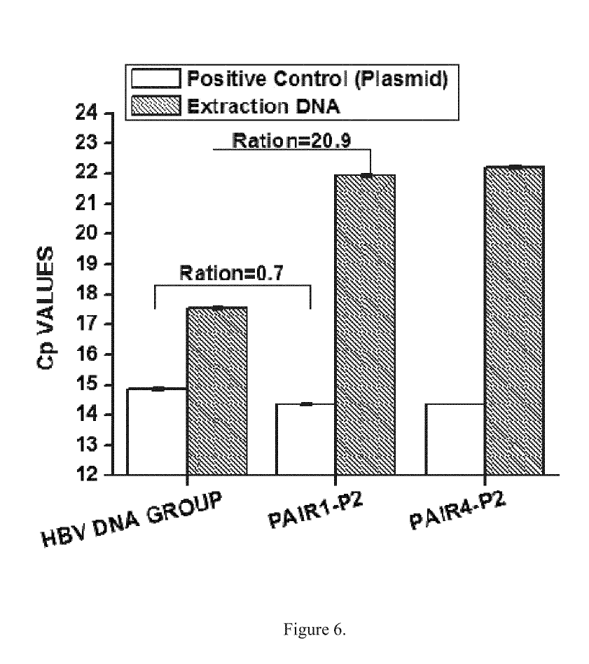

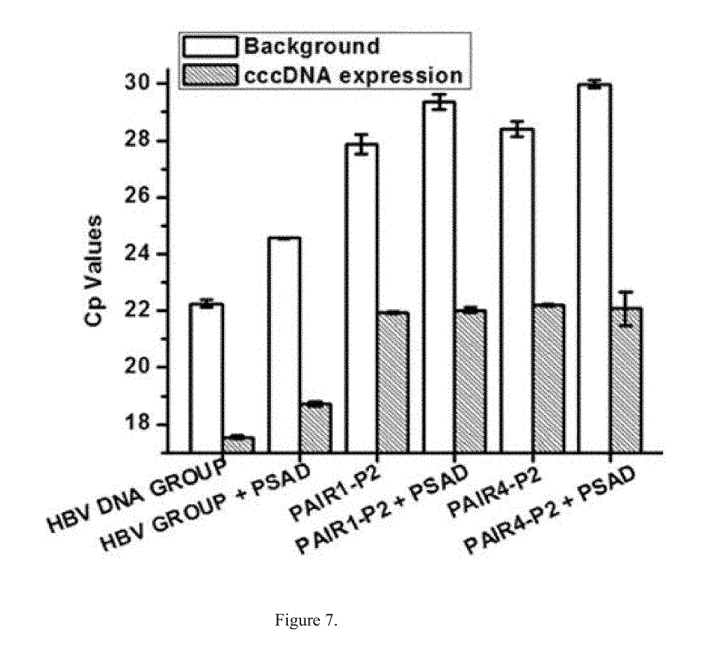

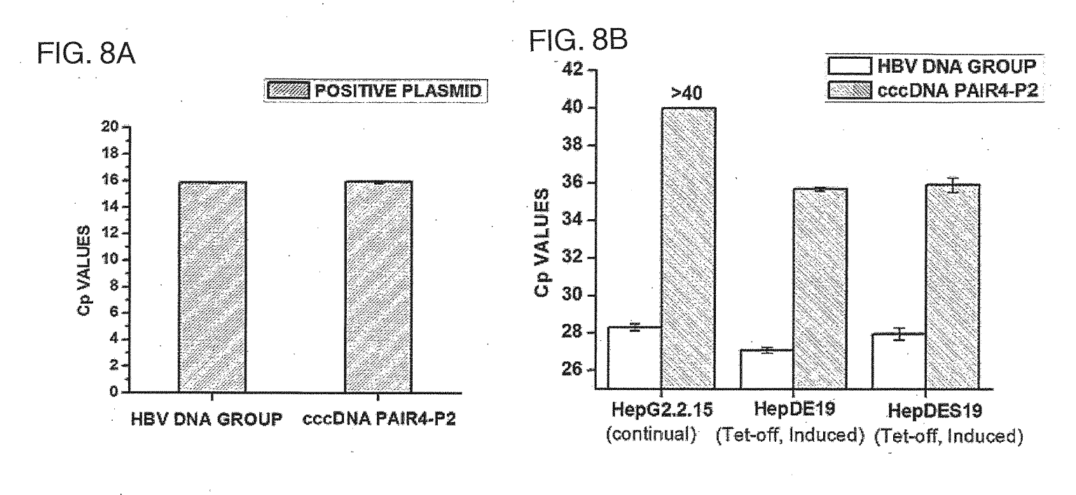

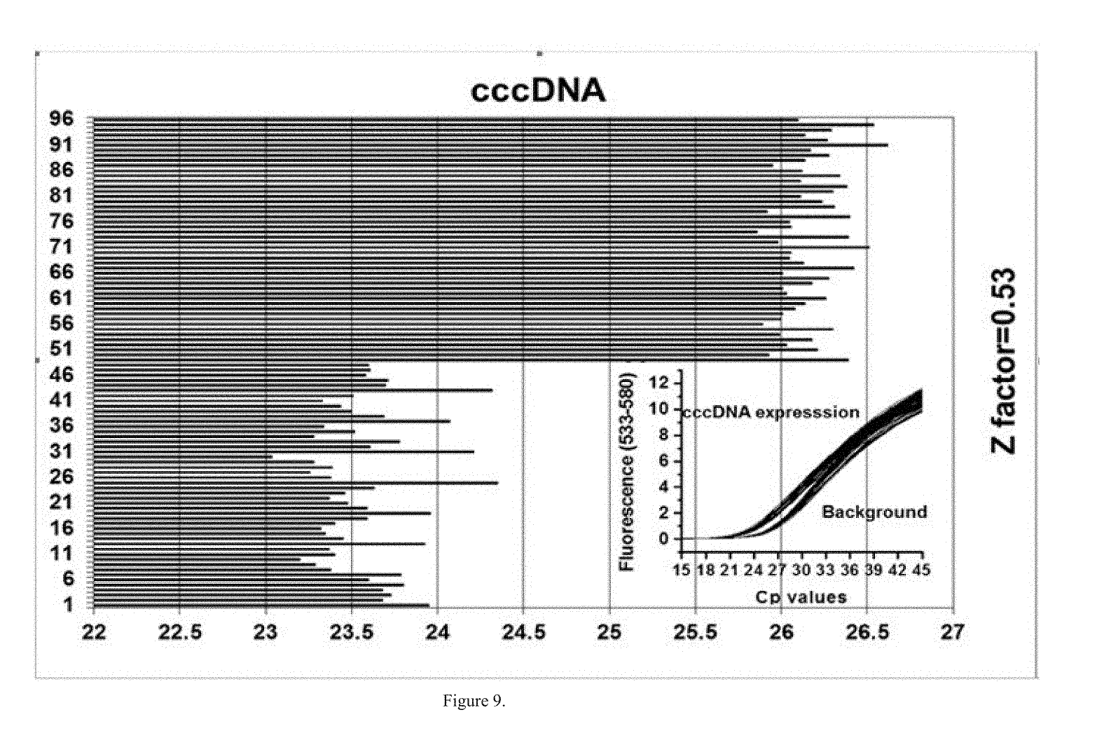

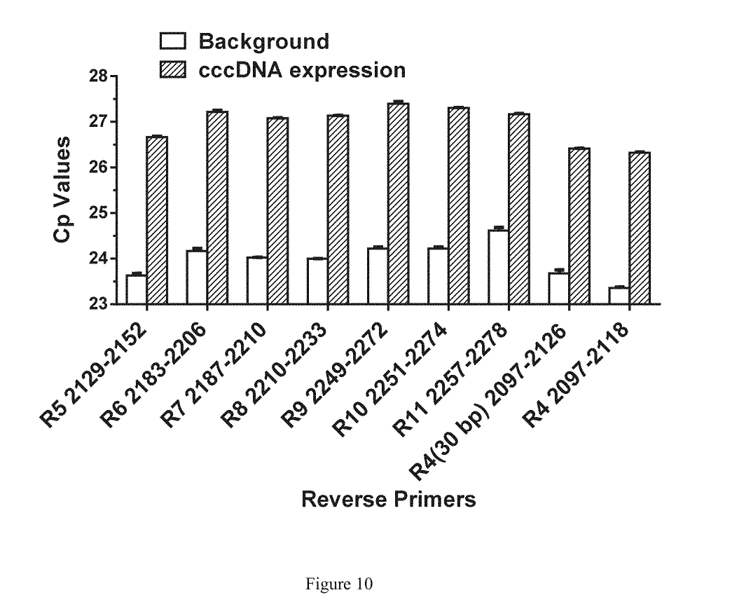

Another embodiment of present invention (xxviii) is principle of new Primers and probes for detecting HBV cccDNA by real-time PCR: the general principle of the selective primers and qPCR design are as shown in FIG. 1. By carefully design qPCR primers at strategic locations of the 1.1×HBV integrated genome, it is possible to generate specific sized qPCR products when using cccDNA as the PCR DNA source while no productive PCR products will result when using the integrated genomic HBV DNA as the PCR source. This primer pairs afford sufficient specificity and selectivity that it is not necessary to use purified viral DNA preparation as the source for qPCR analysis.

### Materials and Methods

### Cell Culture:

HepG2.2.15 cells were cultured at 37° C. in DMEM/F12 medium (Invitrogen, Catalog Number: 10565-018) supplemented with 10% (v/v) fetal bovine serum (Clontech, Tet system Approved FBS, Catalog Number: 631106) and 300 μg/mL G418 (Invitrogen, Catalog Number: 10131-027).

HepDE19 and HepDES19 cells were cultured at 37° C. in DMEM/F12 medium supplemented with 10% (v/v) fetal bovine serum, 300 μg/mL G418, and 1 μg/mL tetracycline (sigma, Catalog Number: T0600000). To obtain HBV viral DNA, HepDES19 cells were seeded in 60 mm culture dish, in DMEM/F12 medium supplemented with 10% (v/v) fetal bovine serum, 300 μg/mL G418 without tetracycline.

### DNA Extraction Method (Modified Hirt DNA Extraction, 1998, Biotechniques 24:760-762):

Cells were resuspended in 250 μL of 50 mM Tris-HCL pH 7.5 and 10 mM EDTA with or without 50 μg/mL RNaseA (Sigma, Catalogue number: R6148). The samples were then mixed with 250 μL of 1.2% SDS (Sigma, Catalog number: 74255) by vortexing and then stood for 5 minutes. Cellular debris and chromosomal DNA were precipitated by the addition of 350 μL of 3 M CsCl, 1 M potassium acetate and 0.67 M acetic acid. The mixture was transferred to a 1.5 mL tube, and then the tube was immediately but gently mixed by pipette and placed on ice for 15 minutes. After the sample was centrifuged for 15 minutes at 14000 g (rcf) at 4° C., the supernatant was loaded onto a column (Qiagen Spin Column; Qiagen GmbH, Hilden, Germany, from QIAamp DNA Mini Kit, Catalogue number: 51306). The column was washed with 750 μL of wash buffer (80 mM potassium acetate, 10 mM Tris, pH7.5, 40 μM EDTA and 60% ethanol V/V) by centrifugation at 14000 g (rcf) at 4° C. DNA was then eluted with 50 μL water or TE buffer (10 mM Tris-HCl, pH 8.0, 1 mM EDTA) by centrifugation at 14000 g (rcf) at 4° C.

One-Step qPCR Assay:

After 40,000 cells per well in 96 well plate were incubated at 37° C. for 6 days, the cells was washed by Ca²⁺ and Mg²⁺ free phosphate-buffered saline (PBS) and lysed by 50 μL of special lysis buffer (Invitrogen, Catalogue Number: AM8723). The resulting sample was incubated for 2 hours at 75° C. in an oven. The lysate was kept at 4° C. until the real time PCR was performed. To perform the qPCR, 2 μL of lysate was used for analysis. For different samples, each reaction was performed in a final volume of 20 μL contained 750 nM of each primer, 400 nM probe and 10 μL of LightCycler 480 Probes Master (RD04887301001-01, Roche) by LightCycler 480 II machine (Roche Diagnositic, INC). The PCR cycling program was performed as follows: started at 50° C. for 2 minutes, then denatured at a temperature of 95° C. for 10 minutes, followed by 45 cycles of amplification at a denaturation temperature of 95° C. for 10 seconds, an annealing temperature of 58° C. for 5 seconds, 63° C. for 10 seconds, and an extension temperature of 72° C. for 40 seconds, and followed by a final extension at a temperature of 72° C. for 10 minutes. After completion of the PCR cycling program, the Cp value was obtained to determine the quantification of input target nucleic acids.

### Examples

The following examples are offered to illustrate, but not to limit the claimed invention.

Abbreviations used herein are as follows:

Conventional Group: means the Conventional Forward Primer (SEQ ID NO: 3) paired with the Conventional Reverse Primer (SEQ ID NO: 4) and the Conventional Probe (5′-TAMRA+SEQ ID NO: 2+BHQ2-3′), “Conventional Group” is abbreviated to “Conventional” in all the figures.

HBV DNA Group: means HBV-F (SEQ ID NO: 25) paired with HBV-R (SEQ ID NO: 26) and HBV DNA Probe (5′-TAMRA+SEQ ID NO: 27+BHQ2-3′)
P0: 5′-dye+SEQ ID NO: 6+quencher-3′
P1: 5′-dye+SEQ ID NO: 7+quencher-3′
P2: 5′-dye+SEQ ID NO: 8+quencher-3′
P3: 5′-dye+SEQ ID NO: 9+quencher-3′
PAIR0: the New Forward cccDNA Primer (SEQ ID NO: 5) paired with R0 (SEQ ID NO: 10)
PAIR1: the New Forward cccDNA Primer (SEQ ID NO: 5) paired with R1 (SEQ ID NO: 11)
PAIR2: the New Forward cccDNA Primer (SEQ ID NO: 5) paired with R2 (SEQ ID NO: 12)
PAIR3: the New Forward cccDNA Primer (SEQ ID NO: 5) paired with R3 (SEQ ID NO: 13)
PAIR4: the New Forward cccDNA Primer (SEQ ID NO: 5) paired with R4 (SEQ ID NO: 14)

### Example 1: Comparison of qPCR Assay Using Conventional cccDNA Primer Pairs for Extracted Viral Genome DNA and Using One-Step qPCR Assay

The sources of cellular materials were from HepDES19 without induction of cccDNA (background due to the presence of 1 μg/mL tetracycline) or with induction (cccDNA expression, by not having tetracycline present) after six days of culture. Conventional Group was tested. DNA Extraction method and one-Step qPCR assay were carried out following procedure described above.

The result shown in FIG. 2 depicted the range between DNA from Tet-on (background) and DNA from Tet-off (cccDNA expression) conditions. For extracted DNA method, the Conventional Group showed 11.3 folds difference in qPCR assay. For one-step qPCR assay, there was only 1.3 folds difference. By comparison of the result of extracted DNA method and one-step qPCR assay, Conventional Group couldn't be used to establish a one-step cccDNA quantification assay.

### Example 2: Test of New cccDNA Primers for Detecting the Extracted DNA or the Direct Lysate without Extraction (One-Step qPCR Assay) from HepDES19

The sources of cellular materials were from HepDES19 without induction of cccDNA (background, due to the presence of 1 μg/mL tetracycline) or with induction (cccDNA expression, by not having tetracycline present) after six days of culture. The following materials were used in this example: Conventional Group, PAIR0-P0, PAIR1-P0, PAIR2-P0, PAIR3-P0 and PAIR4-P0. The dye used in P0 was TAMRA and the quencher used in P0 was BHQ2. One-step qPCR assay was carried out following procedure described above.

The result shown in FIG. 3a depicted that the range between extracted DNA from background and extracted DNA from cccDNA expression in HepDES19 cells was improved from 11.3 folds in the Conventional Group to 50-80 folds in PAIR0-P0, PAIR1-P0, PAIR2-P0, PAIR3-P0 and PAIR4-P0. This indicated the new primer pairs and the probe, namely PAIR0-P0, PAIR1-P0, PAIR2-P0, PAIR3-P0 and PAIR4-P0, had better sensitivity for the detection of cccDNA with low background. The result shown in FIG. 3b depicted that for one-step qPCR assay, there was nearly 6.8 folds difference between background and cccDNA expression. It was worth noticing that, different from Conventional Group, the new cccDNA primer pairs and the probe, namely PAIR0-P0, PAIR1-P0, PAIR2-P0, PAIR3-P0 and PAIR4-P0, could not detect the HBV genome DNA in HepDES19 cells and it was useful for one-step qPCR assay. This explained the improvement associated with the new prime pairs and the probe.

### Example 3: Test of New cccDNA Probes for Detecting the Extracted DNA or the Direct Lysate of One-Step qPCR Assay without Extraction (One-Step qPCR Assay) from HepDES19

The sources of cellular materials were from HepDES19 without induction of cccDNA (background, due to the presence of 1 μg/mL tetracycline) or with induction (cccDNA expression, due to the absence of tetracycline) after six days of culture.

For DNA extraction study, the probes used were P0, P1, P2 and P3, wherein in each probe the dye used was TAMRA and the quencher used was BHQ2. The primer used for all reactions was PAIR4.

For the one-step qPCR assay, the probe used was P2, and the dye used in P2 was TAMRA and the quencher used in P2 was BHQ2. The primers used were PAIR1 and PAIR4 respectively. Conventional Group was also used for control.

The result shown in FIG. 4a depicted the design of new probes and comparison of their ability to detect HBV cccDNA in extracted viral DNA. PAIR4 was fixed in qPCR assay, P0 and P2 showed good efficiency and they had nearly 90 folds difference between background and cccDNA expression. For one-step qPCR assay, the result shown in FIG. 4b depicted that PAIR1-P2 and PAIR4-P2 had more than 8 folds difference, while there was only 1.3 folds difference for Conventional Group. From this result, it is concluded that the new primers and probe pairs are useful for the development of high-throughput assays.

### Example 4: Size Comparison of PCR Products from pHBV1.3 Positive Control Plasmid and One-Step qPCR Assay by DNA Agarose Gel

The sources of cellular materials were from HepDES19 with induction (cccDNA expression, without tetracycline) after six days of culture.

PAIR1-P2, PAIR4-P2 and Conventional Group used in the DNA Extraction and one-step qPCR assay. HBV DNA Group and Conventional Group were used as control. The dye in P2 used was TAMRA and the quencher in P2 used was BHQ2.

The Left part of FIG. 5 was the 1% agarose gel electrophoresis result of reaction product from qPCR using pHBV1.3 as template. The right part of FIG. 5 gave the result of the one-step qPCR assay using the same primer and probe notation.

The result shown in FIG. 5 depicted the size of PCR products from pHBV1.3 positive control plasmid and from one-step qPCR assay were the same, consistent with the PCR product from one-step being the right product. One-step qPCR product from PAIR4-P2 had more specific band than PCR product from the conventional primer pairs.

### Example 5: Confirmation of the Specificity of cccDNA Primers and Probes

The sources of cellular materials were from HepDES19 with induction (cccDNA expression without tetracycline) after six days of culture. pHBV 1.3 plasmid was used as a positive control and the HBV DNA was extracted by the DNA extraction method. HBV DNA Group, PAIR1-P2 and PAIR4-P2 were used in this study. For P2, the dye used was TAMRA and the quencher used was BHQ2. The positive control pHBV1.3 plasmid was used to calibrate the level of various DNA primer pairs. After calibration, the prime pairs generated equal amount of the PCR product (white bars). However, when the same amount of primer pairs was applied to HepDES19 extracted DNA, different Cp values of qPCR products were detected. As shown in FIG. 6, the level of HBV DNA detected in the experiment using HBV DNA Group was 20 folds higher than the level of cccDNA detected in the experiment using PAIR1-P2 and PAIR4-P2 in HepDES19 cells.

### Example 6: Confirmation of the Specificity of cccDNA Primer Pairs

HBV DNA Group, PAIR1-P2 and PAIR4-P2 were used in this study following the procedure of DNA Extraction method. The reaction mixture contained 6.8 μL of extracted DNA, 4U PSAD, 1 mM ATP and 2 μL of reaction buffer with water to final volume of 20 μL. The digestion was carried out at 30° C. for 16 hours. The sources of cellular materials were from HepDES19 without induction of cccDNA (background, with 1 μg/mL tetracycline) or with induction (cccDNA expression, without tetracycline) after six days of culture. The source DNA was extracted DNA from HepDES19 cells. PSAD can cleave non-circular HBV DNA including double-stranded relaxed circular DNA (rcDNA). The result was shown in FIG. 7. There were 2.4 folds difference between the DNA samples with and without PSAD treatment using general HBV DNA Group. However, there was no difference using PAIR1-P2 and PAIR4-P2, indicating the qPCR template for PAIR1-P2 and PAIR4-P2 was a circular source of DNA (cccDNA).

### Example 7: Confirmation of the Specificity of cccDNA Primers Using the Supernatant of HepG2.2.15, HepDE19 and HepDES19 Cells by qPCR Assay

PAIR4-P2 was used in this example. Positive control plasmid pHBV1.3 was used to calibrate the efficiency of HBV DNA primer and cccDNA primer. HepG2.2.15 cells were cultured at 37° C. in DMEM/F12 medium supplemented with 10% (v/v) fetal bovine serum and 300 μg/mL G418. HepDE19 and HepDES19 cells were cultured at 37° C. in DMEM/F12 medium supplemented with 10% (v/v) fetal bovine serum, 300 μg/mL G418, and 1 μg/mL tetracycline. cccDNA was not detected in the supernatant of HepG2.2.15 cells (Stable cell line, continually expression of HBV viral). In the supernatant of HepDE19 cells (six days after tetracycline off) and HepDES19 (six days after tetracycline off) cells, cccDNA was detected only at background level, but rcDNA was detected in these cell lines. After the cells were incubated for 6 days, to perform the qPCR, 2 μL of the supernatant was used for analysis. For different samples, each reaction was performed in a final volume of 20 μL contained 750 nM of each primer, 400 nM of the probe, and 10 μL of LightCycler 480 Probes Master (RD04887301001-01, Roche) by using LightCycler 480 II machine (Roche). The PCR cycling program was performed as follows: started at 50° C. for 2 minutes, then denatured using a temperature of 95° C. for 10 minutes, followed by 45 cycles of amplification at a denaturation temperature of 95° C. for 10 seconds, an annealing temperature of 58° C. for 5 seconds, 63° C. for 10 seconds, and an extension temperature of 72° C. for 40 seconds, and followed by a final extension at a temperature of 72° C. for 10 minutes. The result was shown in FIG. 8.

### Example 8: Assay Development for Detecting HBV cccDNA by Real-Time PCR

PAIR4-P2 was used in this example in one-step qPCR assay. 40,000 cells per well were seeded. The sources of cellular materials were from HepDES19 without induction of cccDNA (background, with 1 μg/mL tetracycline) or with induction (cccDNA expression without tetracycline) after six days of culture. Samples from well 1 to well 48 were cccDNA expression and Samples from 49-96 were background.

The result shown in FIG. 9 depicted the optimized condition. The best incubation of lysis with cell was about half of hour to one hour. The number of the cells on the expression of cccDNA on 96 microtiterplate was 40,000 cells per well. The Cp differential was about 3.0 cycles after Tet off (background) when compared to Tet on (cccDNA expression) and the Z factor was about 0.53, which was suitable for high throughput screening or high throughput screening to screen compounds to block the accumulation of cccDNA in infected live cells.

### Example 9: Comparison of the Ability of New Reverse Primers for Detection of HBV cccDNA by One-Step qPCR Assay

The level of cccDNA was tested in 96 well microtiter plate (Corning 3599) by one-step qPCR assay. In all cases, the New Forward cccDNA primer (SEQ ID NO: 5) and P2 were fixed, wherein the dye in P2 used was TAMRA and the quencher in P2 used was BHQ2. There were also a plurality of the reverse primers were tested, wherein the reverse primer is R5 (SEQ ID NO: 15); R6 (SEQ ID NO: 16); R7 (SEQ ID NO: 17); R8 (SEQ ID NO: 18); R9 (SEQ ID NO: 19); R10 (SEQ ID NO: 20); R11 (SEQ ID NO: 21); or R4(30 bp) (SEQ ID NO: 24). 40,000 cells per well were seeded. The sources of cellular materials were from HepDES19 without induction of cccDNA (background with 1 μg/mL) or with induction (cccDNA expression without tetracycline) after six days of culture. Samples from well 1 to well 48 were cccDNA expression and Samples from 49-96 were background. PAIR4-P2 was used in this assay.

The result was shown in FIG. 10. The reverse primers R5 to R10 showed good efficiency and they had nearly 8 folds difference between background and cccDNA expression. R4 (30 bp) (SEQ ID NO: 24) was also tested. The result in FIG. 10 showed that R4 (30 bp) (SEQ ID NO: 24) was effective similarly compared to R4 (SEQ ID NO: 14).

It is understood that the examples and embodiments described herein are for illustrative purposes only and that various modifications or changes in light thereof will be suggested to persons skilled in the art.

INFORMAL SEQUENCE LISTING

SEQ ID NO: 1: HepDES 19 genomic insert of HBV DNA:
ttccacaaccttccaccaagctcctgcagatcccagagtgagaggcctgt
atttccctgctggtggctccagttcaggaacagtaaaccctgttctgact
actgcctctcccttatcgtcaatcttctcgaggattggggaccctgcgct
gaacatggagaacatcacatcaggattcctaggaccccttctcgtgttac
aggcggggtttttcttgttgacaagaatcctcacaataccgcagagtcta
gactcgtggtggacttctctcaattttctagggggaactaccgtgtgtct
tggccaaaattcgcagtccccaacctccaatcactcaccaacctcttgtc
ctccaacttgtcctggttatcgctggatgtgtctgcggcgttttatcatc
ttcctcttcatcctgctgctatgcctcatcttcttgttggttcttctgga
ctatcaaggtatgttgcccgtttgtcctctaattccaggatcctcaacaa
ccagcacgggaccatgccggacctgcatgactactgctcaaggaacctct
atgtatccctcctgttgctgtaccaaaccttcggacggaaattgcacctg
tattcccatcccatcatcctgggctttcggaaaattcctatgggagtggg
cctcagcccgtttctcctggctcagtttactagtgccatttgttcagtgg
ttcgtagggctttcccccactgtttggctttcagttatatggatgatgtg
gtattgggggccaagtctgtacagcatcttgagtccctttttaccgctgt
taccaattttcttttgtctttgggtatacatttaaaccctaacaaaacaa
agagatggggttactctctaaattttatgggttatgtcattggatgttat
gggtccttgccacaagaacacatcatacaaaaaatcaaagaatgttttag
aaaacttcctattaacaggcctattgattggaaagtatgtcaacgaattg
tgggtcttttgggttttgctgccccttttacacaatgtggttatcctgcg
ttgatgcctttgtatgcatgtattcaatctaagcaggctttcactttctc
gccaacttacaaggcctttctgtgtaaacaatacctgaacctttaccccg
ttgcccggcaacggccaggtctgtgccaagtgtttgctgacgcaaccccc
actggctggggcttggtcatgggccatcagcgcatgcgtggaaccttttc
ggctcctctgccgatccatactgcggaactcctagccgcttgttttgctc
gcagcaggtctggagcaaacattatcgggactgataactctgttgtccta
tcccgcaaatatacatcgtttccatggctgctaggctgtgctgccaactg
gatcctgcgcgggacgtcctttgtttacgtcccgtcggcgctgaatcctg
cggacgacccttctcggggtcgcttgggactctctcgtccccttctccgt
ctgccgttccgaccgaccacggggcgcacctctctttacgcggactcccc
gtctgtgccttctcatctgccggaccgtgtgcacttcgcttcacctctgc
acgtcgcatggagaccaccgtgaacgcccaccaaatattgcccaaggtct
tacataagaggactcttggactctcagcaatgtcaacgaccgaccttgag
gcatacttcaaagactgtttgtttaaagactgggaggagttgggggagga
gattaggttaaaggtctttgtactaggaggctgtaggcataaattggtct
gcgcaccagcaccatgcaactttttcacctctgcctaatcatctcttgtt
catgtcctactgttcaagcctccaagctgtgccttgggtggctttggggc
atggacatcgaccatataaagaatttggagctactgtggagttactctcg
tttttgccttctgacttctttccttcagtacgagatcttctagataccgc
ctcagctctgtatcgggaagccttagagtctcctgagcattgttcacctc
accatactgcactcaggcaagcaattctttgctggggggaactaatgact
ctagctacctgggtgggtgttaatttggaagatccagcgtctagagacct
agtagtcagttatgtcaacactaatatgggcctaaagttcaggcaactct
tgtggtttcacatttcttgtctcacttttggaagagaaacagttatagag
tatttggtgtctttcggagtgtggattcgcactcctccagatatagacca
ccaaatgcccctatcctatcaacacttccggagactactgttgttagacg
acgaggcaggtcccctagaagaagaactccctcgcctcgcagacgaaggt
ctcaatcgccgcgtcgcagaagatctcaatctcgggaatctcaatgttag
tattccttggactcataaggtggggaactttactgggattattatctact
gtacctgtattaatcctcattggaaaacaccatcttttcctaatatacat
ttacaccaagacattatcaaaaaatgtgaacagtttgtaggcccactcac
agttaatgagaaaagaagattgcaattgattatgcctgccaggttttatc
caaaggttaccaaatatttaccattggataagggtattaaaccttattat
ccagaacatctagttaatcattacttccaaactagacactatttacacac
tctatggaaggcgggtatattatataagagagaaacaacacatagcgcct
cattttgtgggtcaccatattcttgggaacaagatctacagcatggggca
gaatattccaccagcaatcctctgggattattcccgaccaccagttggat
ccagccttcagagcaaacaccgcaaatccagattgggacttcaatcccaa
caaggacacctggccagacgccaacaaggtaggagctggagcattcgggc
tgggtttcaccccaccgcacggaggccttttggggtggagccctcaggct
cagggcatactacaaactttgccagcaaatccgcctcctgcctccaccaa
tcgccagtcaggaaggcagcctaccccgctgtctccacctttgagaaaca
ctcatcctcaggccatgcagtggaa
SEQ ID NO: 2: 5′-CGTCGCATGGARACCACCGTGAACGCC-3′
SEQ ID NO: 3: Conventional Forward Primer 1545-
1563″
5′-CTCCCCGTCTGTGCCTTCT-3′
SEQ ID NO: 4: Conventional Reverse Primer 1883-
1900 (reverse complement)
5′-GCCCCAAAGCCACCCAAG-3′
SEQ ID NO: 5: New Forward cccDNA Primer 1528-1548
5′-ACCTCTCTTTACGCGGACTCC-3′
SEQ ID NO: 6: 1617-1641
5′-ACCGTGAACGCCCACCAAATATTGC-3′
SEQ ID NO: 7: 1813-1841
5′-CATGCAACTTTTTCACCTCTGCCTAATCA-3′
SEQ ID NO: 8: 1793-1814
5′-ATTGGTCTGCGCACCAGCACCA-3′
SEQ ID NO: 9: 1855-1881
5′-TCCTACTGTTCAAGCCTCCAAGCTGTG-3′
SEQ ID NO: 10: R0 2005-2025 (reverse complement)
5′-AAGGCTTCCCGATACAGAGCT-3′
SEQ ID NO: 11: R1 1996-2016 (reverse complement)
5′-CGATACAGAGCTGAGGCGGTA-3′
SEQ ID NO: 12: R2 2001-2021 (reverse complement)
5′-CTTCCCGATACAGAGCTGAGG-3′
SEQ ID NO: 13: R3 2046-2067 (reverse complement)
5′-CTGAGTGCAGTATGGTGAGGTG-3′
SEQ ID NO: 14: R4 2097-2118 (reverse complement)
5′-CCCACCCAGGTAGCTAGAGTCA-3′
SEQ ID NO: 15: R5 2129-2152 (reverse complement)
5′-TAGGTCTCTAGACGCTGGATCTTC-3′
SEQ ID NO: 16: R6 2183-2206 (reverse complement)
5′-CCACAAGAGTTGCCTGAACTTTAG-3′
SEQ ID NO: 17: R7 2188-2210 (reverse complement)
5′-GAAACCACAAGAGTTGCCTGAAC-3′
SEQ ID NO: 18: R8 2210-2233 (reverse complement)
5′-TCCAAAAGTGAGACAAGAAATGTG-3′
SEQ ID NO: 19: R9 2249-2272 (reverse complement)
5′-CACTCCGAAAGACACCAAATACTC
SEQ ID NO: 20: R10 2251-2274 (reverse complement)
5′-CACACTCCGAAAGACACCAAATAC-3′
SEQ ID NO: 21: R11 2257-2278 (reverse complement)
5′-AATCCACACTCCGAAAGACACC-3′
SEQ ID NO: 24: R4 (30 bp) 2097-2126 (reverse
complement)
5′-AATTAACACCCACCCAGGTAGCTAGAGTCA-3′
SEQ ID NO: 25: HBV-F: 5′-AAGAAAAACCCCGCCTGTAA-3′
SEQ ID NO: 26: HBV-R:
5′-CCTGTTCTGACTACTGCCTCTCC-3′
SEQ ID NO: 27: 5′-CCTGATGTGATGTTCTCCATGTTCAGC-3′

1. A reverse primer comprising at its extreme 3′ end a sequence recognizing at least 16 consecutive sequence within the nucleotide sequence of the Hepatitis B Virus (HBV) genome (SEQ ID NO: 1).

2. A reverse primer according to claim 1, wherein the reverse primer comprises at its extreme 3′ end a sequence recognizing at least 16 consecutive sequence within nucleotide sequence of HBV genome from 1996 to nucleotide 2278 of SEQ ID NO: 1.

3. A reverse primer according to claim 1 wherein the length of the reverse primer is 16 to 200 nucleotides.

4. A reverse primer according to claim 1, wherein the reverse primer in the 5′ to 3′ direction comprises the sequence selected from:
SEQ ID NO: 10: R0 2005-2025 (reverse complement)
5′-AAGGCTTCCCGATACAGAGCT-3′;
SEQ ID NO: 11: R1 1996-2016 (reverse complement)
5′-CGATACAGAGCTGAGGCGGTA-3′;
SEQ ID NO: 12: R2 2001-2021 (reverse complement)
5′-CTTCCCGATACAGAGCTGAGG-3′;
SEQ ID NO: 13: R3 20452046-2067 (reverse
complement)
5′-CTGAGTGCAGTATGGTGAGGTG-3′;
SEQ ID NO: 14: R4 2097-2118 (reverse complement)
5′-CCCACCCAGGTAGCTAGAGTCA-3′;
SEQ ID NO: 15: R5 2129-2152 (reverse complement)
5′-TAGGTCTCTAGACGCTGGATCTTC-3′;
SEQ ID NO: 16: R6 2183-2206 (reverse complement)
5′-CCACAAGAGTTGCCTGAACTTTAG-3′;
SEQ ID NO: 17: R7 2188-2210 (reverse complement)
5′-GAAACCACAAGAGTTGCCTGAAC-3′;
SEQ ID NO: 18: R8 2210-2233 (reverse complement)
5′-TCCAAAAGTGAGACAAGAAATGTG-3′;
SEQ ID NO: 19: R9 2249-2272 (reverse complement)
5′-CACTCCGAAAGACACCAAATACTC-3′;
SEQ ID NO: 20: R10 2251-2274 (reverse complement)
5′-CACACTCCGAAAGACACCAAATAC-3′;
SEQ ID NO: 21: R11 2257-2278 (reverse complement)
5′-AATCCACACTCCGAAAGACACC-3′;
and
SEQ ID NO: 24: R4 (30 bp) 2097-2126 (reverse
complement)
5′-AATTAACACCCACCCAGGTAGCTAGAGTCA-3′.

5. A primer pair, consisting of the reverse primer of claim 1 and a universal forward primer, wherein the length of the universal forward primer is 16 to 200 nucleotides and the universal forward primer recognizes upstream of DR2 of the HBV genome.

6. A primer pair according to claim 5, wherein the universal forward primer has at least 80% sequence identity with the nucleotide sequence of SEQ ID NO: 5 from nucleotide 1528 to nucleotide 1548.

7. A primer pair according to claim 5, wherein the universal forward primer in the 5′ to 3′ direction comprises SEQ ID NO: 5.

8. A probe for detecting the extracted DNA or the lysate of one-step qPCR assay, wherein the length of the probe is 16 to 200 nucleotides.

9. A probe according to claim 8, wherein the probe has at least 80% sequence identity with a sequence comprising a part of a sequence selected from SEQ ID NO: 6, SEQ ID NO: 7, SEQ ID NO: 8 and SEQ ID NO: 9.

10. A probe according to claim 8, wherein the probe has at least 80% sequence identity with a sequence selected from SEQ ID NO: 6, SEQ ID NO: 7, SEQ ID NO: 8 and SEQ ID NO: 9.

11. A probe according to claim 8, wherein the probe has in the 5′ to 3′ direction the sequence selected from SEQ ID NO: 6, SEQ ID NO: 7, SEQ ID NO: 8 and SEQ ID NO: 9.

12. A probe according to claim 8 wherein the probe is selected from P0, P1, P2 and P3 and wherein:
P0 is 5′-dye+SEQ ID NO: 6+quencher-3′;
P1 is 5′-dye+SEQ ID NO: 7+quencher-3′;
P2 is 5′-dye+SEQ ID NO: 8+quencher-3′;
P3 is 5′-dye+SEQ ID NO: 9+quencher-3′.

13. A combination comprising of a reverse primer and a probe, wherein the reverse primer is selected from R0, R1, R2, R3, R4, R5, R6, R7, R8, R9, R10, R11 and R4 (30 bp), and the probe is selected from P0, P1, P2 and P3.

14. A combination according to claim 13, wherein the reverse primer is selected from R3, R4, R5 and R4 (30 bp) and the probe is selected from TAMRA+SEQ ID NO: 6+BHQ2 and TAMRA+SEQ ID NO: 8+BHQ2.

15. A method for detecting HBV cccDNA by PCR, comprising of using the HBV genome (SEQ ID NO: 1) as the template and using the primer pair of claim 5.

16. A method according to claim 15, wherein the PCR is real-time PCR.

17. A method according to claim 15 wherein the method is a high-throughput method.

18. A one-step cccDNA detection method by real-time PCR without pre DNA extraction and purification, wherein the method comprises the following procedure:
(1) treating cells with compounds to be tested;
(2) harvesting cells after treatment;
(3) adding the lysis buffer to the harvested cells; and
(4) carrying out the real time PCR of claim 15.

19. A method according to claim 18, wherein the lysis buffer is from Invitrogen and the catalogue number of is AM8723.

20. A method according to claim 18 wherein the cells are treated for 30 minutes to 2 hours at a temperature between 50° C. and 75° C. in an oven, 20-100 μL of the lysis buffer is added per well in 96 well plate, and then real time PCR is conducted at a temperature between 0° C. and 25° C.

21. A method used in evaluation of therapeutic effect on HBV comprising using the method of claim 15.

22. A kit for evaluation of therapeutic effect on HBV, said kit comprising the primer pair of claim 5.

23. A kit for evaluation of therapeutic effect on HBV, said kit comprising the combination of claim 13.

24. A method for detecting HBV cccDNA by PCR, comprising of using the HBV genome (SEQ ID NO: 1) as the template and using the combination of claim 13.


SEQUENCE LISTING (27 sequences)

SEQ ID NO: 1 - DNA, 3182 bp, Hepatitis B Virus
ttccacaaccttccaccaagctcctgcagatcccagagtgagaggcctgtatttccctgctggtggctccagttcaggaacagtaaaccctgttctgactactgcctctcccttatcgtcaatcttctcgaggattggggaccctgcgctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatccagcgtctagagacctagtagtcagttatgtcaacactaatatgggcctaaagttcaggcaactcttgtggtttcacatttcttgtctcacttttggaagagaaacagttatagagtatttggtgtctttcggagtgtggattcgcactcctccagcttatagaccaccaaatgcccctatcctatcaacacttccggagactactgttgttagacgacgaggcaggtcccctagaagaagaactccctcgcctcgcagacgaaggtctcaatcgccgcgtcgcagaagatctcaatctcgggaatctcaatgttagtattccttggactcataaggtggggaactttactgggctttattcttctactgtacctgtctttaatcctcattggaaaacaccatcttttcctaatatacatttacaccaagacattatcaaaaaatgtgaacagtttgtaggcccactcacagttaatgagaaaagaagattgcaattgattatgcctgccaggttttatccaaaggttaccaaatatttaccattggataagggtattaaaccttattatccagaacatctagttaatcattacttccaaactagacactatttacacactctatggaaggcgggtatattatataagagagaaacaacacatagcgcctcattttgtgggtcaccatattcttgggaacaagatctacagcatggggcagaatctttccaccagcaatcctctgggattctttcccgaccaccagttggatccagccttcagagcaaacaccgcaaatccagattgggacttcaatcccaacaaggacacctggccagacgccaacaaggtaggagctggagcattcgggctgggtttcaccccaccgcacggaggccttttggggtggagccctcaggctcagggcatactacaaactttgccagcaaatccgcctcctgcctccaccaatcgccagtcaggaaggcagcctaccccgctgtctccacctttgagaaacactcatcctcaggccatgcagtggaa

SEQ ID NO: 2 - DNA, 27 bp, Artificial sequence
  OTHER INFORMATION: Synthetic
cgtcgcatggaraccaccgtgaacgcc

SEQ ID NO: 3 - DNA, 19 bp, Artificial sequence
  OTHER INFORMATION: Synthetic
ctccccgtctgtgccttct

SEQ ID NO: 4 - DNA, 18 bp, Artificial sequence
  OTHER INFORMATION: Synthetic
gccccaaagccacccaag

SEQ ID NO: 5 - DNA, 21 bp, Artificial sequence
  OTHER INFORMATION: Synthetic
acctctctttacgcggactcc

SEQ ID NO: 6 - DNA, 25 bp, Artificial sequence
  OTHER INFORMATION: Synthetic
accgtgaacgcccaccaaatattgc

SEQ ID NO: 7 - DNA, 29 bp, Artificial sequence
  OTHER INFORMATION: Synthetic
catgcaactttttcacctctgcctaatca

SEQ ID NO: 8 - DNA, 22 bp, Artificial sequence
  OTHER INFORMATION: Synthetic
attggtctgcgcaccagcacca

SEQ ID NO: 9 - DNA, 27 bp, Artificial sequence
  OTHER INFORMATION: Synthetic
tcctactgttcaagcctccaagctgtg

SEQ ID NO: 10 - DNA, 21 bp, Artificial sequence
  OTHER INFORMATION: Synthetic
aaggcttcccgatacagagct

SEQ ID NO: 11 - DNA, 21 bp, Artificial sequence
  OTHER INFORMATION: Synthetic
cgatacagagctgaggcggta

SEQ ID NO: 12 - DNA, 21 bp, Artificial sequence
  OTHER INFORMATION: Synthetic
cttcccgatacagagctgagg

SEQ ID NO: 13 - DNA, 22 bp, Artificial sequence
  OTHER INFORMATION: Synthetic
ctgagtgcagtatggtgaggtg

SEQ ID NO: 14 - DNA, 22 bp, Artificial sequence
  OTHER INFORMATION: Synthetic
cccacccaggtagctagagtca

SEQ ID NO: 15 - DNA, 24 bp, Artificial sequence
  OTHER INFORMATION: Synthetic
taggtctctagacgctggatcttc

SEQ ID NO: 16 - DNA, 24 bp, Artificial sequence
  OTHER INFORMATION: Synthetic
ccacaagagttgcctgaactttag

SEQ ID NO: 17 - DNA, 23 bp, Artificial sequence
  OTHER INFORMATION: Synthetic
gaaaccacaagagttgcctgaac

SEQ ID NO: 18 - DNA, 24 bp, Artificial sequence
  OTHER INFORMATION: Synthetic
tccaaaagtgagacaagaaatgtg

SEQ ID NO: 19 - DNA, 24 bp, Artificial sequence
  OTHER INFORMATION: Synthetic
cactccgaaagacaccaaatactc

SEQ ID NO: 20 - DNA, 24 bp, Artificial sequence
  OTHER INFORMATION: Synthetic
cacactccgaaagacaccaaatac

SEQ ID NO: 21 - DNA, 22 bp, Artificial sequence
  OTHER INFORMATION: Synthetic
aatccacactccgaaagacacc

SEQ ID NO: 22-23 - skipped (2 entries)

SEQ ID NO: 24 - DNA, 30 bp, Artificial sequence
  OTHER INFORMATION: Synthetic
aattaacacccacccaggtagctagagtca

SEQ ID NO: 25 - DNA, 20 bp, Artificial sequence
  OTHER INFORMATION: Synthetic
aagaaaaaccccgcctgtaa

SEQ ID NO: 26 - DNA, 23 bp, Artificial sequence
  OTHER INFORMATION: Synthetic
cctgttctgactactgcctctcc

SEQ ID NO: 27 - DNA, 27 bp, Artificial sequence
  OTHER INFORMATION: Synthetic
cctgatgtgatgttctccatgttcagc